(19) United States
(12) Patent Application Publication
(10) Pub. No.: US 2023/0310196 A1
(43) Pub. Date: Oct. 5, 2023

(54) WEARABLE DEVICES AND METHODS OF MANUFACTURE AND USE THEREOF

(71) Applicant: Sheldon Harper, East Orange, NJ (US)

(72) Inventor: Sheldon Harper, East Orange, NJ (US)

(21) Appl. No.: 18/127,499

(22) Filed: Mar. 28, 2023

Related U.S. Application Data
(60) Provisional application No. 63/325,199, filed on Mar. 30, 2022.

(51) Int. Cl. A61F 5/01

(52) U.S. Cl. CPC A61F 5/0125

(57) ABSTRACT

Disclosed are wearable devices and methods of manufacture and use thereof. In particular, these technologies enable treatment and prevention of injuries to the knee joint of the wearer by stabilizing the knee joint of the wearer, in particular providing mediolateral support to the wearer, or by providing compression support to the leg of the wearer.

### CROSS-REFERENCE TO RELATED PATENT APPLICATION

This patent application claims a benefit of priority to U.S. Provisional Patent Application 63/325,199, filed on Mar. 30, 2022, which is hereby incorporated by reference in its entirety for all purposes.

### TECHNICAL FIELD

This disclosure relates to wearable devices and methods of manufacture and use thereof.

### BACKGROUND

The knee is one of the largest joints in the human body, and includes bones, cartilage, muscles, ligaments, and tendons. The knee is commonly known to be one of the most easily injured joints and is vulnerable to a variety of injuries and problems. Some of the most common knee problems and injuries include tendinopathy (tendonitis), sprains, ligament tears, cartilage tears, arthritis dislocations, and fractures.

Bracing is commonly used as a form of orthotic intervention for knee injuries and problems. However, most conventional knee braces are unable to simultaneously provide adequate knee support and muscle support to the entire leg, without slipping from the leg while being worn on the leg.

### SUMMARY

Generally, this disclosure enables various wearable devices and methods of manufacture and use thereof. In particular, these technologies enable treatment and minimization or prevention of injuries to the knee joint of the wearer by stabilizing the knee joint of the wearer, in particular providing mediolateral support to the wearer, and by providing compression support to the leg of the wearer. For example, some embodiments of the wearable device may provide support and stability to an injured knee of the wearer suffering from ligament sprain or tear, or osteoarthritis, where the wearable device may aid in muscle support to the entire leg of the wearer, while providing a more comfortable and stable wear to the wearer by minimizing or omitting slipping by including an upper body and foot feature. For example, the wearable device may enable balance and weight shifting management to the wearer through the added upper body shoulder strap, ensuring added support to the leg of the wearer, and aiding in further slipping stoppage of the brace and leg support component. For example, the wearable device may offer some aid in pain relief to the wearer due to the further weight shifting management.

In an embodiment, a device comprises: a shoulder strap including a first end portion and a second end portion; a waist belt including a frontal portion and a back portion, wherein the first end portion extends from the frontal portion and the second end portion extends from the back portion such that the shoulder strap spans between the waist belt when a user having a shoulder, a waist, a leg, and a knee (a) wears the shoulder strap over the shoulder and (b) extends the waist belt around the waist; and a leg portion defined by an upper leg portion, a lower leg portion, and a knee brace, wherein the knee brace spans between the upper leg portion and the lower leg portion, wherein the upper leg portion extends from the waist belt such that the upper leg portion spans between the waist belt and the knee brace and the waist portion spans between the shoulder strap and the upper leg portion when (a) the leg extends through the upper leg portion, the lower leg portion, and the knee brace, (b) the knee brace engages the knee, (c) the upper leg portion and the lower leg portion collectively provide a compression support for the leg and (d) the knee brace provides a mediolateral stability to the knee.

In an embodiment, a method comprises: causing a user to access a device including a shoulder strap, a waist belt, and a leg portion, wherein the user includes a shoulder, a waist, a leg, and a knee, wherein the shoulder strap includes a first end portion and a second end portion, wherein the waist belt includes a frontal portion and a back portion, wherein the leg portion is defined by an upper leg portion, a lower leg portion, and a knee brace, wherein the knee brace spans between the upper leg portion and the lower leg portion; and causing the user to wear the device such that (a) the first end portion extends from the frontal portion and the second end portion extends from the back portion such that the shoulder strap spans between the waist belt, (b) the shoulder strap extends over the shoulder, (c) the waist belt extends around the waist, (d) the upper leg portion extends from the waist belt, (e) the upper leg portion spans between the waist belt and the knee brace, (f) the waist portion spans between the shoulder strap and the upper leg portion, (g) the leg extends through the upper leg portion, the lower leg portion, and the knee brace, (h) the knee brace engages the knee, (i) the upper leg portion and the lower leg portion collectively provide a compression support for the leg, and (j) the knee brace provides a mediolateral stability to the knee.

In an embodiment, a method comprises: supplying a device to a user, wherein the device includes a shoulder strap, a waist belt, and a leg portion, wherein the user includes a shoulder, a waist, a leg, and a knee, wherein the shoulder strap includes a first end portion and a second end portion, wherein the waist belt includes a frontal portion and a back portion, wherein the is leg portion defined by an upper leg portion, a lower leg portion, and a knee brace, wherein the knee brace spans between the upper leg portion and the lower leg portion; and instructing the user to wear the device such that (a) the first end portion extends from the frontal portion and the second end portion extends from the back portion such that the shoulder strap spans between the waist belt, (b) the shoulder strap extends over the shoulder, (c) the waist belt extends around the waist, (d) the upper leg portion extends from the waist belt, (e) the upper leg portion spans between the waist belt and the knee brace, (f) the waist portion spans between the shoulder strap and the upper leg portion, (g) the leg extends through the upper leg portion, the lower leg portion, and the knee brace, (h) the knee brace engages the knee, (i) the upper leg portion and the lower leg portion collectively provide a compression support for the leg, and (j) the knee brace provides a mediolateral stability to the knee.

### DESCRIPTION OF DRAWINGS

FIG. 1 is a front view of an embodiment of a wearable device in a closed position according to this disclosure.

FIG. 2 is a rear view of an embodiment of a wearable device in a closed position according to this disclosure.

FIG. 3 is a side view of an embodiment of a wearable device in a closed position according to this disclosure.

FIG. 4 is a front view of an embodiment of a wearable device in an open position according to this disclosure.

FIG. 5 is a rear view of an embodiment of a wearable device in an open position according to this disclosure.

FIG. 6 is a side view of an embodiment of a wearable device in an open position according to this disclosure.

FIG. 7A is a front view of an embodiment of a wearable device in a closed position as would be worn by a human wearer according to this disclosure.

FIG. 7B is a rear view of an embodiment of a wearable device in a closed position as would be worn by a human wearer according to this disclosure.

### DETAILED DESCRIPTION

Generally, this disclosure enables various wearable devices and methods of manufacture and use thereof. In particular, these technologies enable support and stabilization of the knee joint of the wearer secondary to ligament injury of the wearer, postoperative reconstruction of the wearer, meniscus damage of the wearer, and for preventative protection of the wearer. For example, some embodiments of the wearable device are designed to treat the range of maladies of the wearer that can affect the knee joint of the wearer.

This wearable device may be medically beneficial in various scenarios. For example, the wearable device may provide a solution to knee injury and ailments suffered by many. It may be a better choice for support and stability for those wishing to get more from a knee brace by providing support and stability to an injured knee suffering from ligament sprain or tear, cartilage tears, arthritis, dislocations, and fractures. The wearable device may carry added features to aid in muscle support to the entire leg. The wearable device may provide comfortable and stable wear by omitting slipping of the component by including a shoulder strap, waist belt and foot strap for the wearer. Balance and weight shifting management to the wearer may be delivered through the waist belt and shoulder strap of the wearer, ensuring added support to the leg of the wearer, and aiding in stopping slipping of the brace and leg support component. The design offers some aid in pain relief to the wearer due to weight shifting management. The wearable device may not only provide support and stability to the wearer, but may also aid in the prevention of knee injuries to the wearer.

The knee joint comprises three bones known as the femur, patella, and tibia. The quadricep and patella tendon connect the knee bones to the leg muscles, providing movement of the knee joint, while cruciate and collateral ligaments join the knee bones and provide stability to the knee. The joint surfaces (the ends of the femur and tibia, and the back of the patella) are covered by articular cartilages that aids the knee bones to glide smoothly across each other as the leg is bent or straightened. Resting on top of the tibial cartilage are the medial and lateral meniscus acting as shock absorbers between the femur and tibia, improving stability of the joint and helping to distribute weight. Working all together the proper form of flexion and extension as well as lateral and medial rotation of the knee joint is permitted and performed. The specific components of the knee joint, and certain maladies that can have an effect on them, are discussed in greater detail below. In addition, the benefits of certain features of various embodiments of the presently disclosed wearable device are discussed.

Tendons

The quadriceps tendon connects the muscles in the front of the thigh to the patella, working alongside the muscles in the front of the thigh so that the leg can be straightened. The patellar tendon runs from the patella to the tibia and works with the muscles at the front of the thigh to extend the knee to enable kicking, running, and jumping. The quadriceps and patellar tendons may experience tendinopathy and can also be stretched and torn, making it difficult to walk or carry out other everyday activities.

Tendinopathy is where tendons around the knee become painful and swollen, restricting movement. Tendinopathy is usually caused by overuse (through activities such as running, jumping, or cycling) or repeated minor accidents. Tendinopathy causes irritation and inflammation of one or more tendons. Quadriceps tendinopathy affects the attachment of the quadriceps tendon to the top of the kneecap (patella) and is less common than patella tendinopathy. Rupture of the quadricep tendon may be a partial tear or a complete tear. This is where the muscle is separated from the kneecap so that knee can no longer straighten when the quadriceps muscle tightens. Patellar tendinopathy, also called jumper's knee, occurs when the force of hitting the ground after a jump strains the patellar tendon. This in turn leads to tendon weakness and, in some cases, tiny tears in the tendon. This tendon can be torn where it attaches to the bottom of the kneecap either partially or completely. For non-surgical treatment of both quadricep and patellar tendon tears, wearing of a brace is normally recommended.

The presently disclosed wearable device utilizes an upper and lower body system designed to support weight bearing. The upper body part consists of a waist belt with a shoulder strap that further adds to the stability of the leg and aids in weight shifting management. The leg support portion of the device includes an upper and lower cross strapping system. The upper portion of the leg support runs from the top of the knee brace to the waste belt, supporting the muscles that surround the femur, while the lower portion runs from the bottom of the knee brace to the foot, supporting the muscles that surround the tibia and fibula. The presently disclosed wearable device is designed to aid in off-loading weight bearing loads that affect the knee tendons, ligaments, and bones. This is especially useful for pain relief within the knee joint that is associated with injury.

Cruciate Ligaments

The cruciate ligaments (anterior cruciate ligament (ACL) and posterior cruciate ligament (PCL)) control the back-and-forth motion of the knee and are responsible for a large part of the knee's stability. Both the ACL and PCL can be injured due to stress and/or trauma to the knee. One of the most common knee injuries is the rupturing of the ACL, whereas PCL injuries are less common. ACL injury typically occurs during movements with high knee valgus moments in combination with internal or external rotations of the tibia, and may require surgical repair. Whether or not surgical treatment is taken, a brace is normally recommended to protect the knee from instability.

Depending on the severity of the injury to the ACL, reconstruction of the ligament is undertaken, where the damaged ligament is replaced with a tissue graft. The new ACL will be weaker than the native (original) ACL during the first 12 months, so a brace during this initial period helps protect it from harmful forces that occur in everyday activities.

Certain embodiments of the wearable device are designed with a brace body including a stretchable, breathable fabric material, with front and rear bands that secure a comfortable and supportive fit to the knee. Adding to the front and rear bands are guided upper and lower strappings with ends that wrap around the knee at the femur and the tibia. The upper and lower knee area strappings aid in the prevention of excessive joint movement and improve stability during activity, and thus prevent secondary injuries. In some embodiments, the outer and inner sides of the brace body include removable hinged joints (which in certain instances can be removable) that move internally upon flexion, providing added stability while replicating the motion of the knee.

The presently disclosed wearable device generates relative motion between the tibia and femur closely resembling normal knee motion when the orthosis is in use. This transfers force from the anterior tibia to the distal posterior femur and aids in resisting internal rotation of the tibia on the femur, replicating the functions of the ACL. This also aids in the prevention of injury to the secondary structures if not already damaged, because often injuries to the anterior cruciate ligament occur along with damage to other structures in the knee, such as articular cartilage, meniscus, or other ligaments.

Collateral Ligaments

The medial collateral ligament (MCL) and lateral collateral ligament (LCL) are located on the inner and outer side of the knee joint and restrict the joint from translating extreme or excessive mediolateral movement.

Particular embodiments of the presently disclosed wearable device include outer and inner hinged joints at the sides of the brace body. These play an important role in stabilizing the knee joint in the case of collateral ligament rupture. In the case of an MCL and or PCL rupture, the hinged joints aid in controlling the sideways motion of the knee joint, thus preventing the knee from buckling both inward and outward. The hinged joints also prevent MCL and or PCL injury.

Menisci

Meniscus tears are among the most common knee injuries. The menisci help to transmit weight from one bone to another, and play a vital role in the knee's stability. A forceful twist or rotation of the knee, especially when bearing full weight on it, can lead to a torn meniscus. This leads to a feeling of the knee joint to be catching or locking (feeling as though the knee is locked in place when trying to move it), or a sensation of the knee giving way. Meniscal tears may also occur as a result of aging. As people age, they are more likely to have degenerative meniscus tears. However, anyone at any age can tear the meniscus. Meniscus tears are less commonly required to have surgical treatment but instead it is normally recommended to wear a brace during an activity to protect the knee from further injury.

Some embodiments of the presently disclosed wearable device include front and rear bands that wrap around with the upper and lower knee strapping system, and outer and inner hinged joints. These features provide support to control instability at the front and sides of the knee thus preventing extra strain on the meniscus. This takes the pressure off the meniscus, allowing it to rest. The elastic material, band and strapping system also performs as a compression bandage, delivering compression to prevent additional swelling and blood loss. The wearable device supports the knee's recovery after meniscal tear surgery or during a conservative (non-surgical) treatment program. The extra support, stability, and compression helps decrease swelling, which reduces pain and promotes healing within the knee. Without this kind of intervention, it is more likely to develop osteoarthritis in the injured knee.

Arthritis

Knee arthritis is inflammation and deterioration of the knee joint cartilage causing pain, swelling, and stiffness. Knee arthritis is a serious disability for many people, making it hard to do many everyday activities, such as walking or climbing stairs. There are many types of arthritis, but the most common types are osteoarthritis and rheumatoid arthritis. Osteoarthritis is the most common form of arthritis, and often affects the knees. It is a degenerative process where the cartilage in the joint gradually deteriorates and wears away. This is normally caused by aging and wear and tear or excess stress on the joint such as repeated injury or being overweight. Rheumatoid arthritis is a chronic autoimmune disease, where the immune system attacks its own tissues. Rheumatoid arthritis can affect the knees by causing the joint to become inflamed and by destroying the knee cartilage. Rheumatoid arthritis often affects persons at an earlier age than osteoarthritis. If untreated, rheumatoid arthritis can cause permanent joint damage.

The presently disclosed wearable device is designed to help support the entire knee load. This aids in decreasing compressive loads transmitted to the joint surfaces, creating stability, and supporting the joint to help take pressure off it. This results in the diminishing of pain associated with knee arthritis and increases function of the knee, improving the ability to get around and help walk comfortably.

Dislocation and Fractures

Other Injuries that may occur within the knee are dislocation and/or fractures of the bones. Knee dislocation is where the femur and tibia lose contact with each other. The femur and tibia can be forced out of alignment, and the patella can also slip out of place. The patella, also known as the kneecap, is a small bone at the front of the knee joint. The location of the patella at the front of the knee makes it vulnerable to fracture, dislocation, or tendon tears. Fractures of the knee are caused by high energy trauma (falls or auto accidents). The most common bone broken around the knee is the patella.

Certain embodiments of the presently disclosed wearable device includes a sleeve/wrap around hybrid design that includes elastic material with a patellar cutout and buttress, front and rear bands, and strappings at the knee area. These features are suited to treat patellar mal-tracking and tendon swelling. The wearable device compresses the knee joint, improving blood flow, increases warmth, and stabilizes the patella. The patellar cutout and buttress help stabilize the patella and promote proper patellar tracking, while the strapping system at the lower part of the knee brace portion applies pressure to the patellar tendon reducing tendon strain and also provides stability. The removable outer and inner hinged joints at the sides of the knee brace portion also help with joint instability and aids in preventing future injury or degeneration.

Muscle

Common debilitating effects of knee injury includes decreased muscle size and strength. If the muscle joint lacks in receiving proper physical activity in the daily routine, the muscles will start to weaken. Muscle disuse due to lower limb injury, such as ACL tear and other knee injuries, can impair both muscle morphology and function. Weak muscles are a leading cause of knee injuries. Quadriceps, Hamstrings and Gastrocnemius are the most important muscles that are responsible for the knee stability. In the cases where these muscles get weakened, it may lead to conditions like a looser knee joint, which could lead to knee weakness and buckling. A lack of strength and flexibility can increase the risk of knee injuries.

Embodiments of the presently disclosed wearable device include an upper and lower leg support strap system, which aids in supporting the leg muscles, functioning as a reinforcement for the weakened muscles due to knee injury or added strength for normally functioning muscles, aiding in the prevention of knee injury. The wearable device gives the wearer the confidence to conduct daily activities that remedy the inactivity that leads to muscle weakness. The knee brace is comfortable, light weight, and remains in perfect alignment at all times, providing added stability and compression to protect the knee.

Note that this disclosure may be embodied in many different forms and should not be construed as necessarily being limited to various embodiments disclosed herein. Rather, these embodiments are provided so that this disclosure is thorough and complete, and fully conveys various concepts of this disclosure to skilled artisans. The drawings illustrate example embodiments and are not to be construed as necessarily limiting this disclosure. Like numbers or similar numbering schemes can refer to like or similar elements throughout.

Various terminology used herein can imply direct or indirect, full or partial, temporary or permanent, action or inaction. For example, when an element is referred to as being “on,” “connected,” or “coupled” to another element, then the element can be directly on, connected, or coupled to another element or intervening elements can be present, including indirect or direct variants. In contrast, when an element is referred to as being “directly connected” or “directly coupled” to another element, then there are no intervening elements present.

As used herein, various singular forms “a,” “an” and “the” are intended to include various plural forms (e.g., two, three, four, five, six, seven, eight, nine, ten, tens, hundreds, thousands) as well, unless specific context clearly indicates otherwise.

As used herein, various presence verbs “comprises,” “includes” or “comprising,” “including” when used in this specification, specify a presence of stated features, integers, steps, operations, elements, or components, but do not preclude the presence or addition of one or more other features, integers, steps, operations, elements, components, or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

As used herein, a term “or others,” “combination”, “combinatory,” or “combinations thereof” refers to all permutations and combinations of listed items preceding that term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. Skilled artisans understand that typically there is no limit on number of items or terms in any combination, unless otherwise apparent from the context.

As used herein, unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized or overly formal sense unless expressly so defined herein.

As used herein, relative terms such as “below,” “lower,” “above,” “upper,” “left” and “right” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below. Similarly, if a device in one of illustrative figures were turned over, then various elements described as “left” or “right” of other elements would then be oriented “right” or “left” of the other elements.

As used herein, a term “about” or “substantially” refers to a +/−10% variation from a nominal value/term. Such variation is always included in any given value/term provided herein, whether or not such variation is specifically referred thereto.

Features described with respect to certain embodiments may be combined in or with various some embodiments in any permutational or combinatory manner. Different aspects or elements of example embodiments, as disclosed herein, may be combined in a similar manner.

Although various terms first, second, third, and so forth can be used herein to describe various elements, components, regions, layers, or sections, these elements, components, regions, layers, or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Features described with respect to certain example embodiments can be combined and sub-combined in or with various other example embodiments. Also, different aspects or elements of example embodiments, as disclosed herein, can be combined and sub-combined in a similar manner as well. Further, some example embodiments, whether individually or collectively, can be components of a larger system, wherein other procedures can take precedence over or otherwise modify their application. Additionally, a number of steps can be required before, after, or concurrently with example embodiments, as disclosed herein. Note that any or all methods or processes, at least as disclosed herein, can be at least partially performed via at least one entity in any manner.

Example embodiments of this disclosure are described herein with reference to illustrations of idealized embodiments (and intermediate structures) of this disclosure. As such, variations from various illustrated shapes as a result, for example, of manufacturing techniques or tolerances, are to be expected. Thus, various example embodiments of this disclosure should not be construed as necessarily limited to various particular shapes of regions illustrated herein, but are to include deviations in shapes that result, for example, from manufacturing.

Any or all elements, as disclosed herein, can be formed from a same, structurally continuous piece, such as being unitary, or be separately manufactured or connected, such as being an assembly or modules. Any or all elements, as disclosed herein, can be manufactured via any manufacturing processes, whether additive manufacturing, subtractive manufacturing, or other any other types of manufacturing. For example, some manufacturing processes include three dimensional (3D) printing, laser cutting, computer numerical control (CNC) routing, milling, pressing, stamping, vacuum forming, hydroforming, injection molding, lithography, carving, chiseling, and so forth.

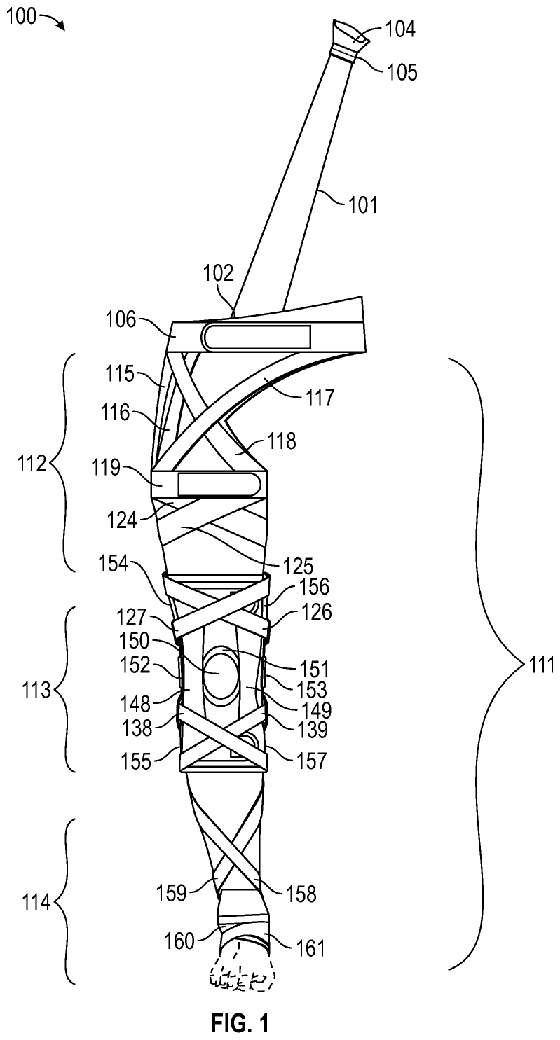

FIG. 1 illustrates a front view of an embodiment of a wearable device in the closed position according to this disclosure. As shown in FIG. 1, a wearable device 100 includes shoulder strap 101 (e.g., functioning like a suspender) having first end 102, shoulder pad 104, means for adjusting shoulder strap 105, waist belt 106, leg portion 111 (e.g., functioning like a leg portion of a pair of pants), having upper leg portion 112, knee brace 113, and lower leg portion 114. For example, the shoulder strap 101 can be a monolithic piece or an assembly of pieces. Upper leg portion 112 includes first upper leg strap 115 (also referred to as central thigh strap), second upper leg strap 116 (also referred to as front thigh strap), third upper leg strap 117 (also referred to as cross-thigh to waist strap), fourth upper leg strap 118 (also referred to as front inner thigh strap), fifth upper leg strap 119 (also referred to as upper thigh strap), sixth upper leg strap 124 (also referred to as upper strap 1), and seventh upper leg strap 125 (also referred to as upper strap 2). Knee brace 113 includes first knee brace strap 126 (also referred to as upper knee strap 1), second knee brace strap 127 (also referred to as upper knee strap 2), third knee brace strap 138 (also referred to as lower knee strap 1), fourth knee brace strap 139 (also referred to as lower knee strap 2), first knee brace band 148 (also referred to as R front band), second knee brace band 149 (also referred to as L front band), open portion 150 (also referred to as patella cut-out), patellar donut buttress 151, first hinged joint support 152 (also referred to as R hinged joint support), second hinged joint support 153 (also referred to as L hinged joint support), first hinged joint support first holder 154 (also referred to as R upper hinge holder), first hinged joint support second holder 155 (also referred to as R lower hinge holder), second hinged joint support third holder 156 (also referred to as L upper hinge holder), and second hinged joint support fourth holder 157 (also referred to as R upper hinge holder). Lower leg portion 114 includes first lower leg strap 158 (also referred to as lower strap 1), second lower leg strap 159 (also referred to as lower strap 2), third lower leg strap 160, and fourth lower leg strap 161.

The waist belt 106 can be a monolithic piece or an assembly of pieces. The waist belt 106 can be monolithic with the shoulder strap 101 (at any end portion thereof) or the leg portion 111 or be assembled with any therewith (e.g., fastening). The waist belt 106 fits around the waist, and generally comprises an elastic material, a resilient material, or a shape memory material with ends including fasteners. Any type of fastener is contemplated for use as fasteners, including, but not limited to, a hook-and-loop fastener, a zipper, a button, or a pair of tie ends. Attached to the waist belt 106 is shoulder strap 101, which extends from the front portion of the waist belt 106, generally more to the side of the leg portion 111 of the device 100, up and over the shoulder opposite of the leg portion 111 and extends from the rear portion of the waist belt 106, generally mirroring the position on the front portion of the waist belt 106, such that the shoulder strap 101 spans between the waist belt when a user having a shoulder, a waist, a leg, and a knee wears the shoulder strap over the shoulder and extends the waist belt around the waist. The shoulder strap 101, and first upper leg strap 115, second upper leg strap 116, third upper leg strap 117, fourth upper leg strap 118, fifth upper leg strap 119, sixth upper leg strap 124, seventh upper leg strap 125, first knee brace strap 126, second knee brace strap 127, third knee brace strap 138, fourth knee brace strap 139, first lower leg strap 158, second lower leg strap 159, third lower leg strap 160, and fourth lower leg strap 161 generally consists of a fabric mesh or webbing material, such as, but not limited to, cotton or canvas, or synthetic polymers, including those that contain polypropylene or polyamides (nylon). In certain embodiments, the shoulder pad 104 can be a slightly padded elastic material that is designed to cushion the shoulder strap 101 to the shoulder while being worn. and the means for adjusting the shoulder strap can be an adjustable buckle or slider buckle used to adjust the length of the strap to fit the wearer more ideally and more comfortably.

The knee brace 113 includes a sleeve body, which can comprise an elastic material, a resilient material, or a shape memory material, generally fits the leg at the knee area, with the top of the knee brace 113 being placed at the lower thigh above the knee joint and the bottom of the knee brace 113 at the calf muscle below the knee joint. The knee brace includes first 148 and second 149 bands, which function generally as a wraparound feature of the knee brace 113. In certain embodiments, the first 148 and second 149 bands include a generally crescent shaped, padded elastic material that are designed to cross each other and be fastened down when being worn. When fastened across each other, some embodiments of the first 148 and second 149 bands leave a central opening for more flexibility of movement at the joint.

At the sides of the knee brace are removable first 152 and second 153 hinged joint supports, which generally comprise a metal (e.g., aluminum or titanium) or a metal alloy (e.g., steel), or can be manufactured from other materials (e.g., plastic, rubber, wood, silicon), or any combination thereof. Each of the first 152 and second 153 hinged joint supports include a central hinge or swivel body and upper and lower portions. The upper and lower portions can each have a cross-section of any generally closed shape, including, but not limited to, generally square, generally rectangular, generally rounded, generally circular, generally ovoid, generally triangular, generally polygonal or generally non-polygonal, or other suitable shapes, or any combination thereof, or generally open shape, including, but not limited to, U-shaped, V-shaped, or other suitable shapes.

The portions of the first hinged joint support 152 and the second hinged joint support 153 on either side of the hinge can be rigid (e.g., unable to be manually bent), but can also be somewhat flexible (e.g., able to be manually bent), or a combination of rigid and somewhat flexible portions. The portions of the first hinged joint support 152 and the second hinged joint support 153 on either side of the hinge can each be monolithic (e.g., a single unit including a same material, additively manufactured, 3-D printed, cast, injection molded), but can also be an assembly of parts (e.g., by fastening, mating, interlocking). The portions of the first hinged joint support 152 and the second hinged joint support 153 on either side of the hinge can each be internally solid (e.g., a bar), but can be hollow (e.g., a cylinder) or compartmentalized (e.g., with a set of compartments).

The upper leg portion 112 spans between the waist belt 106 and the knee brace 113. The upper leg portion 112 includes an elastic material, a resilient material, or a shape memory material interwoven with straps that generally encloses the thigh and its upper leg muscular system. The straps generally provide support for the hip and upper thigh. The lower leg portion 114 spans the bottom of the knee brace 113 to the foot. The lower leg portion includes an elastic material, a resilient material, or a shape memory material interwoven with straps that generally encloses the muscular system of the lower leg and wraps around the foot.

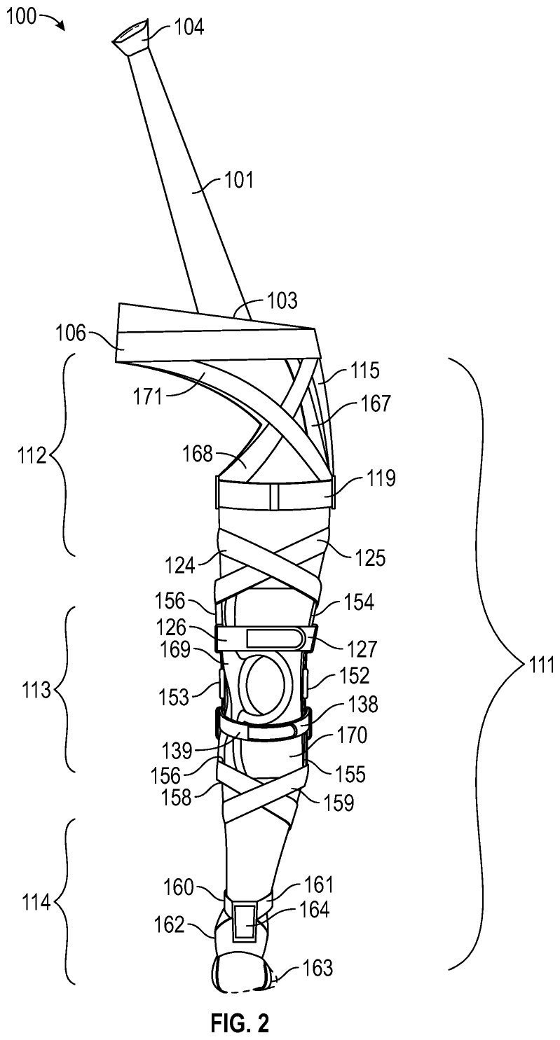

FIG. 2 illustrates a rear view of an embodiment of a wearable device in the closed position according to this disclosure. As shown in FIG. 2, the wearable device 100 includes shoulder strap 101 having second end 103, shoulder pad 104, waist belt 106, leg portion 111, having upper leg portion 112, knee brace 113, and lower leg portion 114. Upper leg portion 112 includes first upper leg strap 115 (also referred to as central thigh strap), eighth upper leg strap 167 (also referred to as rear thigh strap), ninth upper leg strap 168 (also referred to as rear inner thigh strap), tenth upper leg strap 171 (also referred to as rear cross-thigh to waist strap), fifth upper leg strap 119 (also referred to as upper thigh strap), sixth upper leg strap 124 (also referred to as upper strap 1), and seventh upper leg strap 125 (also referred to as upper strap 2). Knee brace 113 includes first knee brace strap 126 (also referred to as upper knee strap 1), second knee brace strap 127 (also referred to as upper knee strap 2), third knee brace strap 138 (also referred to as lower knee strap 1), fourth knee brace strap 139 (also referred to as lower knee strap 2), third knee brace band 169 (also referred to as L rear band), fourth knee brace band 170 (also referred to as R rear band), first hinged joint support 152 (also referred to as R hinged joint support), second hinged joint support 153 (also referred to as L hinged joint support), first hinged joint support first holder 154 (also referred to as R upper hinge holder), first hinged joint support second holder 155 (also referred to as R lower hinge holder), second hinged joint support third holder 156 (also referred to as L upper hinge holder), and second hinged joint support fourth holder 157 (also referred to as R upper hinge holder). Lower leg portion 114 includes first lower leg strap 158 (also referred to as lower strap 1), second lower leg strap 159 (also referred to as lower strap 2), third lower leg strap 160, fourth lower leg strap 161, ankle strap 162, foot strap 163 and Achilles support 164.

As was the case with the bands shown in FIG. 1, eighth upper leg strap 167, ninth upper leg strap 168, tenth upper leg strap 171, ankle strap 162, and foot strap 163 generally consists of a fabric mesh or webbing material, such as, but not limited to, cotton or canvas, or synthetic polymers, including those that contain polypropylene or polyamides (nylon). Achilles support 164 generally comprises an elastic material, a resilient material, or a shape memory material, or other materials (e.g., plastic, rubber, or silicon), or any combination thereof.

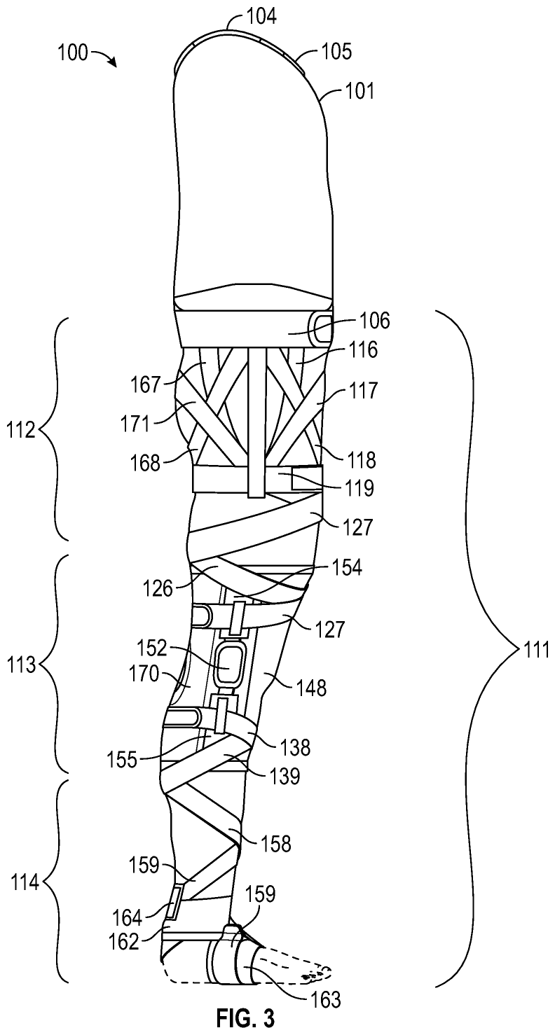

FIG. 3 illustrates a side view of an embodiment of a wearable device in the closed position according to this disclosure. As shown in FIG. 3, wearable device 100 includes shoulder strap 101, shoulder pad 104, means for adjusting shoulder strap 105, waist belt 106, leg portion 111, having upper leg portion 112, knee brace 113, and lower leg portion 114. Upper leg portion 112 includes second upper leg strap 116, third upper leg strap 117, fourth upper leg strap 118, fifth upper leg strap 119, eighth upper leg strap 167, ninth upper leg strap 168, and tenth upper leg strap 171. Knee brace 113 includes first knee brace strap 126, second knee brace strap 127, third knee brace strap 138, fourth knee brace strap 139, first knee brace band 148, second knee brace band 149, first hinged joint support 152, first hinged joint support first holder 154, and first hinged joint support second holder 155. Lower leg portion 114 includes first lower leg strap 158, second lower leg strap 159, ankle strap 162, foot strap 163 and Achilles support 164.

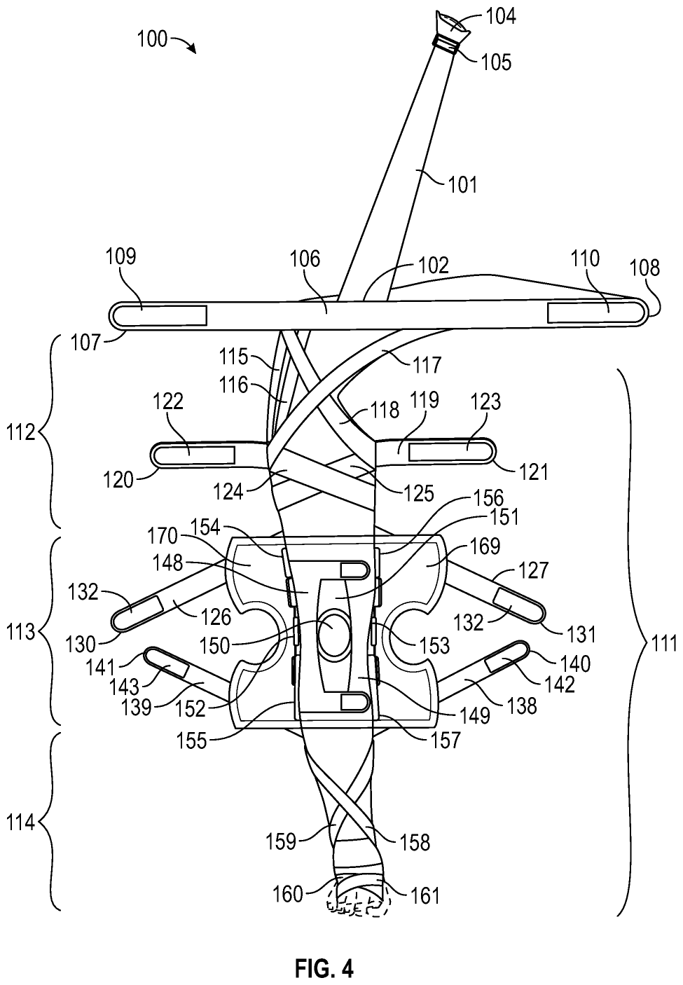

FIG. 4 illustrates a front view of an embodiment of a wearable device in the open position according to this disclosure. As shown in FIG. 4, wearable device 100 includes shoulder strap 101 having first end 102, shoulder pad 104, means for adjusting shoulder strap 105, waist belt 106 having first end 107, second end 108, first fastener 109 and second fastener 110, leg portion 111, having upper leg portion 112, knee brace 113, and lower leg portion 114. Upper leg portion 112 includes first upper leg strap 115, second upper leg strap 116, third upper leg strap 117, fourth upper leg strap 118, fifth upper leg strap 119 having first end 120, second end 121, first fastener 122 and second fastener 123, sixth upper leg strap 124, and seventh upper leg strap 125. Knee brace 113 includes first knee brace strap 126 having first end 130 and first fastener 132, second knee brace strap 127 having second end 131 and second fastener 133, third knee brace strap 138 having first end 140 and first fastener 142, fourth knee brace strap 139 having second end 141 and second fastener 143, first knee brace band 148, second knee brace band 149, open portion 150, patellar donut buttress 151, first hinged joint support 152, second hinged joint support 153, first hinged joint support first holder 154, first hinged joint support second holder 155, second hinged joint support third holder 156, and second hinged joint support fourth holder 157. Lower leg portion 114 includes first lower leg strap 158, second lower leg strap 159, third lower leg strap 160, and fourth lower leg strap 161.

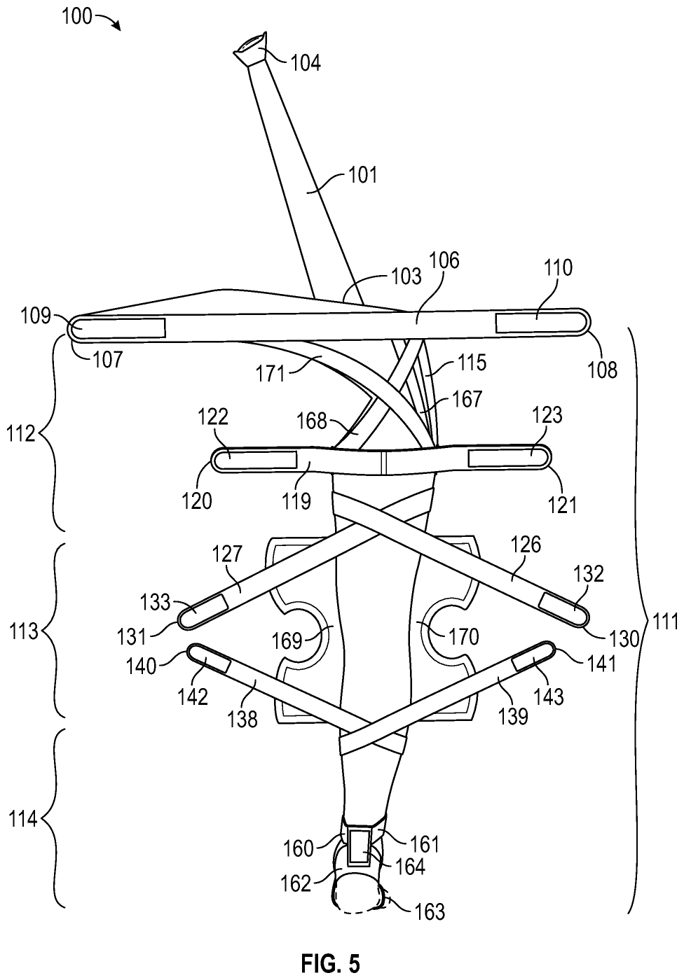

FIG. 5 illustrates a rear view of an embodiment of a wearable device in the open position according to this disclosure. As shown in FIG. 5, knee brace and leg support 100 includes shoulder strap 101 having second end 103, shoulder pad 104, waist belt 106 having first end 107, second end 108, first fastener 109 and second fastener 110, leg portion 111, having upper leg portion 112, knee brace 113, and lower leg portion 114. Upper leg portion 112 includes first upper leg strap 115, eighth upper leg strap 167, ninth upper leg strap 168, tenth upper leg strap 171, and fifth upper leg strap 119 having first end 120, second end 121, first fastener 122 and second fastener 123. Knee brace 113 includes first knee brace strap 126 having first end 130 and first fastener 132, second knee brace strap 127 having second end 131 and second fastener 133, third knee brace strap 138 having first end 140 and first fastener 142, fourth knee brace strap 139 having second end 141 and second fastener 143, third knee brace band 169, and fourth knee brace band 170. Lower leg portion 114 includes third lower leg strap 160, fourth lower leg strap 161, ankle strap 162, foot strap 163 and Achilles support 164.

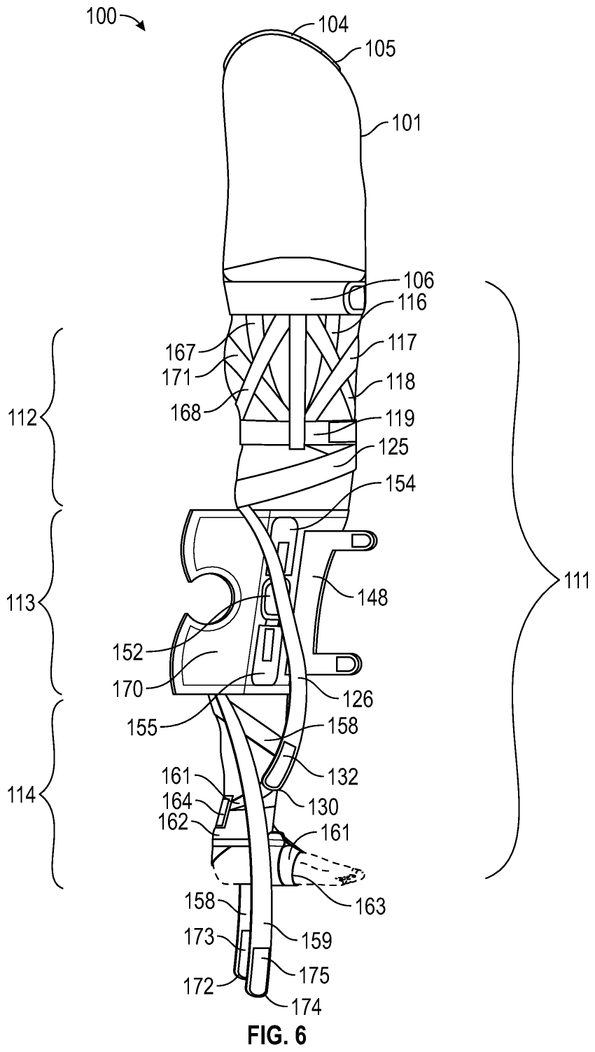

FIG. 6 illustrates a side view of an embodiment of a wearable device in the open position according to this disclosure. As shown in FIG. 6, wearable device 100 includes shoulder strap 101, shoulder pad 104, means for adjusting shoulder strap 105, waist belt 106, leg portion 111, having upper leg portion 112, knee brace 113, and lower leg portion 114. Upper leg portion 112 includes second upper leg strap 116, third upper leg strap 117, fourth upper leg strap 118, fifth upper leg strap 119, eighth upper leg strap 167, ninth upper leg strap 168, and tenth upper leg strap 171. Knee brace 113 includes third knee brace strap 138 having first end 140 and first fastener 142, first knee brace band 148, fourth knee brace band 170, first hinged joint support 152, first hinged joint support first holder 154, and first hinged joint support second holder 155. Lower leg portion 114 includes first lower leg strap 158 having first end 172 and first fastener 173, second lower leg strap 159 having second end 174 and second fastener 175, fourth lower leg strap 161, ankle strap 162, foot strap 163 and Achilles support 164.

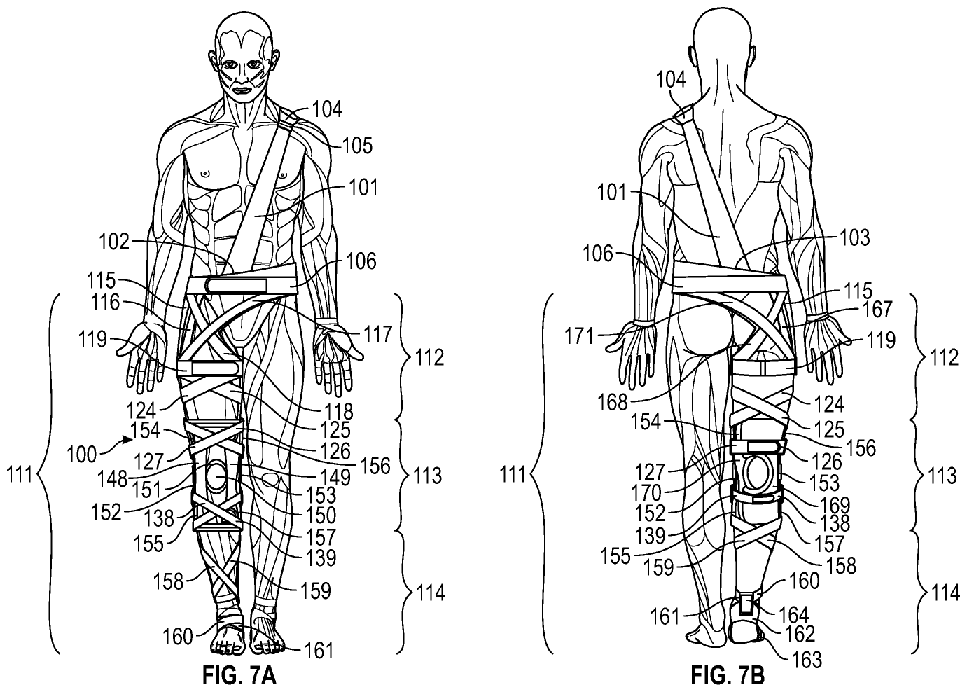

FIG. 7A illustrates a front view of an embodiment of a wearable device in the closed position as would be worn by a human according to this disclosure. As shown in FIG. 7A, wearable device 100 includes shoulder strap 101 having first end 102, shoulder pad 104, means for adjusting shoulder strap 105, waist belt 106, leg portion 111, having upper leg portion 112, knee brace 113, and lower leg portion 114. Upper leg portion 112 includes first upper leg strap 115, second upper leg strap 116, third upper leg strap 117, fourth upper leg strap 118, fifth upper leg strap 119, sixth upper leg strap 124, and seventh upper leg strap 125. Knee brace 113 includes first knee brace strap 126, second knee brace strap 127, third knee brace strap 138, fourth knee brace strap 139 first knee brace band 148, second knee brace band 149, open portion 150, patellar donut buttress 151, first hinged joint support 152, second hinged joint support 153, first hinged joint support first holder 154, first hinged joint support second holder 155, second hinged joint support third holder 156, and second hinged joint support fourth holder 157. Lower leg portion 114 includes first lower leg strap 158, second lower leg strap 159, third lower leg strap 160, and fourth lower leg strap 161.

FIG. 7B illustrates a rear view of an embodiment of a wearable device in the closed position as would be worn by a human according to this disclosure. As shown in FIG. 7B, wearable device 100 includes shoulder strap 101 having second end 103, shoulder pad 104, waist belt 106, leg portion 111, having upper leg portion 112, knee brace 113, and lower leg portion 114. Upper leg portion 112 includes first upper leg strap 115, eighth upper leg strap 167, ninth upper leg strap 168, tenth upper leg strap 171, fifth upper leg strap 119, sixth upper leg strap 124, and seventh upper leg strap 125. Knee brace 113 includes first knee brace strap 126, second knee brace strap 127, third knee brace strap 138, fourth knee brace strap 139, third knee brace band 169, fourth knee brace band 170, first hinged joint support 152, second hinged joint support 153, first hinged joint support first holder 154, first hinged joint support second holder 155, second hinged joint support third holder 156, and second hinged joint support fourth holder 157. Lower leg portion 114 includes first lower leg strap 158, second lower leg strap 159, third lower leg strap 160, fourth lower leg strap 161, ankle strap 162, foot strap 163 and Achilles support 164.

As explained above, the presently disclosed wearable device may be medically beneficial in various scenarios, including, but not limited to, tendinopathy, partial or complete tendon tear, ACL or PCL rupture or injury, MCL or LCL rupture or injury, torn meniscus, arthritis of the knee joint, dislocation and/or fracture of one or more bones in or around the knee, tear of one or muscles in or around the knee, decreased muscle size and strength, or other medical conditions.

Various corresponding structures, materials, acts, and equivalents of all means or step plus function elements in various claims below are intended to include any structure, material, or act for performing the function in combination with other claimed elements as specifically claimed. Various embodiments were chosen and described in order to best disclose various principles of this disclosure and various practical applications thereof, and to enable others of ordinary skill in a pertinent art to understand this disclosure for various embodiments with various modifications as are suited to a particular use contemplated. This detailed description has been presented for various purposes of illustration and description, but is not intended to be fully exhaustive or limited to this disclosure in various forms disclosed. Many modifications and variations in techniques and structures will be apparent to those of ordinary skill in an art without departing from a scope and spirit of this disclosure as set forth in various claims that follow. Accordingly, such modifications and variations are contemplated as being a part of this disclosure. Scope of this disclosure is defined by various claims, which include known equivalents and unforeseeable equivalents at a time of filing of this disclosure.

What is claimed is:

1. A device, comprising:
a shoulder strap including a first end portion and a second end portion;
a waist belt including a frontal portion and a back portion, wherein the first end portion extends from the frontal portion and the second end portion extends from the back portion such that the shoulder strap spans between the waist belt when a user having a shoulder, a waist, a leg, and a knee (a) wears the shoulder strap over the shoulder and (b) extends the waist belt around the waist; and
a leg portion defined by an upper leg portion, a lower leg portion, and a knee brace, wherein the knee brace spans between the upper leg portion and the lower leg portion, wherein the upper leg portion extends from the waist belt such that the upper leg portion spans between the waist belt and the knee brace and the waist portion spans between the shoulder strap and the upper leg portion when (a) the leg extends through the upper leg portion, the lower leg portion, and the knee brace, (b) the knee brace engages the knee, (c) the upper leg portion and the lower leg portion collectively provide a compression support for the leg and (d) the knee brace provides a mediolateral stability to the knee.

2. The device of claim 1, wherein the shoulder strap hosts a shoulder pad and a means for adjusting the shoulder strap, wherein the shoulder pad is hosted between the first end portion and the second end portion, wherein the means for adjusting the shoulder strap is hosted between the first end portion and the second end portion.

3. The device of claim 1, wherein the shoulder strap diagonally opposes the upper leg portion when the user (a) wears the shoulder strap over the shoulder and (b) the leg extends through the upper leg portion, the lower leg portion, and the knee brace.

4. The device of claim 1, wherein the waist belt is fastenable via a strap including at least one of a hook-and-loop fastener, a zipper, a button, or a pair of tie ends.

5. The device of claim 1, wherein the upper leg portion is fastenable via a strap including at least one of a hook-and-loop fastener, a zipper, a button, or a pair of tie ends.

6. The device of claim 1, wherein the knee brace is fastenable via a strap including at least one of a hook-and-loop fastener, a zipper, a button, or a pair of tie ends.

7. The device of claim 1, wherein the lower leg portion is fastenable via a strap including at least a hook-and-loop fastener, a zipper, a button, or a pair of tie ends.

8. The device of claim 1, wherein the knee includes a patella, wherein the knee brace includes an open portion extending at least one of around or over the patella when the knee brace engages the knee.

9. The device of claim 1, wherein the knee brace includes a patellar donut buttress.

10. The device of claim 9, where the knee includes a patella, wherein the patellar donut buttress extends around the patella when the knee brace engages the knee.

11. The device of claim 9, wherein the patella donut buttress is a first patellar donut buttress, wherein the knee brace includes a second patellar donut buttress.

12. The device of claim 1, wherein the leg includes an ankle and a foot, wherein the lower leg portion further includes an ankle strap fastenable around the ankle and a foot strap fastenable around the foot when the leg extends through the upper leg portion, the lower leg portion, and the knee brace.

13. The device of claim 1, wherein the leg includes an Achilles tendon, wherein the lower leg portion further includes a member supporting the Achilles tendon when the leg extends through the upper leg portion, the lower leg portion, and the knee brace.

14. The device of claim 1, wherein the knee portion further includes a hinged joint support, a first holder engaging the hinged joint support, and a second holder engaging hinged joint support.

15. The device of claim 14, wherein the hinged joint support is a first hinged joint support, wherein the knee portion further includes a second hinged joint support, a third holder engaging the second hinged joint support, and a fourth holder engaging the second hinged joint support.

16. The device of claim 14, wherein the hinged joint support includes at least one of a metal or a metal alloy.

17. The device of claim 1, wherein at least one of the upper leg portion, the knee portion, or the lower leg portion comprises at least one of an elastic material, a resilient material, or a shape memory material.

18. The device of claim 17, wherein the elastic material is at least one of breathable or includes a synthetic fiber.

19. A method, comprising:
causing a user to access a device including a shoulder strap, a waist belt, and a leg portion, wherein the user includes a shoulder, a waist, a leg, and a knee, wherein the shoulder strap includes a first end portion and a second end portion, wherein the waist belt includes a frontal portion and a back portion, wherein the leg portion is defined by an upper leg portion, a lower leg portion, and a knee brace, wherein the knee brace spans between the upper leg portion and the lower leg portion; and
causing the user to wear the device such that (a) the first end portion extends from the frontal portion and the second end portion extends from the back portion such that the shoulder strap spans between the waist belt, (b) the shoulder strap extends over the shoulder, (c) the waist belt extends around the waist, (d) the upper leg portion extends from the waist belt, (e) the upper leg portion spans between the waist belt and the knee brace, (f) the waist portion spans between the shoulder strap and the upper leg portion, (g) the leg extends through the upper leg portion, the lower leg portion, and the knee brace, (h) the knee brace engages the knee, (i) the upper leg portion and the lower leg portion collectively provide a compression support for the leg, and (j) the knee brace provides a mediolateral stability to the knee.

20. A method comprising:
supplying a device to a user, wherein the device includes a shoulder strap, a waist belt, and a leg portion, wherein the user includes a shoulder, a waist, a leg, and a knee, wherein the shoulder strap includes a first end portion and a second end portion, wherein the waist belt includes a frontal portion and a back portion, wherein the leg portion is defined by an upper leg portion, a lower leg portion, and a knee brace, wherein the knee brace spans between the upper leg portion and the lower leg portion; and
instructing the user to wear the device such that (a) the first end portion extends from the frontal portion and the second end portion extends from the back portion such that the shoulder strap spans between the waist belt, (b) the shoulder strap extends over the shoulder, (c) the waist belt extends around the waist, (d) the upper leg portion extends from the waist belt, (e) the upper leg portion spans between the waist belt and the knee brace, (f) the waist portion spans between the shoulder strap and the upper leg portion, (g) the leg extends through the upper leg portion, the lower leg portion, and the knee brace, (h) the knee brace engages the knee, (i) the upper leg portion and the lower leg portion collectively provide a compression support for the leg, and (j) the knee brace provides a mediolateral stability to the knee.
